(19) United States
(12) Patent Application Publication
(10) Pub. No.: US 2024/0316029 A1
(43) Pub. Date: Sep. 26, 2024

(54) NEW TREATMENT OF IMMUNODEFICIENCY DISORDER

(71) Applicant: ENLITISA (SHANGHAI) PHARMACEUTICAL CO., LTD., Shanghai (CN)

(72) Inventor: Bengt Ingemar SAMUELSSON, Solna (SE)

(21) Appl. No.: 18/681,447

(22) PCT Filed: Aug. 5, 2022
(86) PCT No.: PCT/CN2022/110573

§ 371 Date: Feb. 5, 2024

(30) Foreign Application Priority Data
Aug. 6, 2021 (WO) PCT/CN2021/111247

(51) Int. Cl. A61K 31/47; A61P 17/02; A61P 29/00; A61P 37/04

(52) U.S. Cl. CPC A61K 31/47; A61P 17/02; A61P 29/00; A61P 37/04

(57) ABSTRACT

The use of montelukast or a pharmaceutically acceptable salt thereof for the manufacture of a medicament for the treatment of an immunodeficiency disorder and also for the treatment of a disorder characterized by inflammation in a patient that has, or is vulnerable to, a condition characterized by immunosuppression. Particular disorders that may be mentioned include those brought on by radiation therapy as part of e.g. cancer treatment, such as radiation proctitis. Montelukast and salts thereof are preferably administered topically and locally, for example anorectally.

### FIELD OF THE INVENTION

This invention relates to a new use of a known pharmaceutically active compound and, in particular, its use in the treatment of conditions characterized by immunosuppression, such as radiation proctitis.

### BACKGROUND AND PRIOR ART

The immune system is the body's natural defence against foreign or dangerous invaders, such as microorganisms (e.g. bacteria, viruses, and fungi), parasites (e.g. worms) and cancer cells.

The immune system distinguishes between what belongs in the body and what does not. Substances that are recognised by the immune system are termed ‘antigens’. In otherwise healthy individuals, antigens stimulate an immune response in the body if perceived as dangerous. The normal sequence of events consists of recognition of the potentially harmful antigen, and then activating the body's immune response to mobilise and neutralise it.

The innate immune system is the dominant and primary immune system response. It operates by recruiting immune cells to a site of infection through the production of chemical mediators (cytokines), activation the complement cascade to identify bacteria, activate cells, and identifying and removing foreign substances present in organs, tissues, blood and lymph.

Cytokines released by injured cells in response to infection or irritation thends to result in inflammation, which serves to establish a physical barrier against the spread of infection and to promote healing of any damaged tissue following the clearance of pathogens.

The acute inflammation stage occurs at the onset of infection or injury, and is initiated by cells present in tissues (e.g. in macrophages, dendritic cells, etc.). These cells undergo activation through one of their pattern recognition receptors (PRRs), and recognize a pathogen-associated molecular pattern (PAMP) to release inflammatory mediators (e.g. histamine, bradykinin, serotonin, leukotrienes and prostaglandins), which sensitize pain receptors, cause local vasodilation, and attract phagocytes (e.g. neutrophils), which trigger other parts of the immune system by releasing factors that summon additional leukocytes and lymphocytes.

The innate immune system also activates the secondary strand of the immune system in higher vertebrates, known as the adaptive immune system. The adaptive immune system is composed of specialized, systemic cells and processes that eliminate pathogens or prevent their growth.

Unlike the innate immune system, the adaptive immune system is specific to each particular pathogen the body has encountered in the past, and creates an immunological memory following an initial response to a specific pathogen. This leads to an enhanced response to future encounters with that pathogen. This is the process by which a person that recovers from a viral or bacterial disease is protected thereafter, sometimes for the rest of their life, and is the basis for the process of vaccination.

The key cells that are involved in the adaptive immune response are two different types of lymphocytes, B cells and T cells. In the adaptive immune response, B cells are activated to secrete antibodies (immunoglobulins), which bind to an antigen and inactivate it, so it cannot cause damage, whereas T cells help identify and destroy foreign or abnormal cells.

Adaptive immunity results in pathogen-specific receptors being acquired to prepare the immune system for future challenges. However, on some occasions, the adaptive immune system is unable to distinguish harmful from harmless foreign bodies, like pollen or food molecules, resulting in allergies or allergic conditions, such as asthma and hay fever, or in autoimmune conditions, in which the immune system may attack the body's own tissues, including rheumatoid arthritis and systemic lupus erythematosus.

Other disorders of the immune system include those in which the body is unable to generate an appropriate immune response against antigens. Such a condition is often termed ‘immunodeficiency’, in which a patient is ‘immunocompromised’.

Disorders characterised by immunodeficiency, or ‘immunodeficiency disorders’ impair the immune system's ability to defend the body against antigens. As a result, infections may occur more easily, or cancers, such as lymphomas, may develop. Many people that have immunodeficiency disorders also have autoimmune disorders.

Primary immunodeficiency disorders (PIDDs) are typically rare, congenital disorders that are usually inherited. Such disorders often, but not always, present themselves during childhood, and may be characterised according to the part of the immune system that is affected (missing, reduced in number, abnormal and/or malfunctioning), including: B cells (humoral immunodeficiency); T cells (cellular immunodeficiency); phagocytes (neutrophils, monocytes, macrophages, and eosinophil; phagocytic immunodeficiency); and/or complement proteins (complement deficiency). Humoral immunodeficiency disorders are the most common PIDDs, accounting for more than half.

Secondary immunodeficiency disorders (SIDDs) are more common and tend to develop later in life. They are usually a consequence of something else, including old age, malnutrition (particularly undernutrition), exposure to chemicals (including drugs), a chronic disease or disorder, such as diabetes, HIV infection or cancer (including leukaemia and lymphoma, which compromise the bone marrow's ability to produce lymphocytes), or chemotherapy and/or radiation therapy, which are used to treat disorders such as cancer.

Active pharmaceutical ingredients are sometimes designed to have an immunosuppressive effect, depending upon what it is that they are intended to treat. Examples include those that are given to prevent rejection of a transplanted organ or tissue, or to patients with autoimmune disorders, as well as corticosteroids, which are often used to suppress inflammation resulting from an over-active immune system in various disorders, such as rheumatoid arthritis. However, an obvious side effect of inducing immunosuppression is that is affects the body's innate ability to fight off infections, as described above.

Current treatments of immunodeficiency disorders are somewhat limited. In addition to prevention of infections through good hygiene, vaccines, antivirals and antibiotics are often given. In addition, replacing parts of the immune system that are missing, such as through immunoglobulin therapy (i.e. treating patients with antibodies obtained from the blood of people with a normal immune system), is sometimes effective.

Severely affected patients often require intensive and frequent treatment throughout life and their conditions can often only by corrected by stem cell transplantation. Gene therapy and thymus tissue transplantation is sometimes helpful, but these are expensive and are reserved for the most life-threatening immunodeficiency disorders.

Hence there is a clear unmet clinical need for more effective and/or more readily available treatments of immunodeficiency disorders.

Of particular interest here is radiation therapy, which works by damaging (e.g. cancer) cells through the direct effect of ionizing radiation on DNA, lipids, and proteins. As water makes up the majority of the cell, ionization radiation results in the creation of oxygen-free radicals (OFRs). The generation of OFRs is known to be involved in the development of the systemic inflammatory response syndrome. OFRs activate (amongst others) cytokine production. Cytokines are the main mediators involved in the progression of systemic inflammatory response (Closa et al, IUBMB Life, 56, 185 2004)).

Radiation proctitis is inflammation of the rectum that occurs as a result of damage to the rectum sustained from pelvic radiation given to treat a cancer such as prostate or cervical cancer. Radiation proctitis can be acute or chronic depending on the timing relative to the radiation therapy, but is essentially a result of radiation doses that result in the normal tissue losing its ability to repair or recover from injury. The etiology is unclear, but it has been suggested that the systemic glutathione deficiency following radiation leads to increased oxidative injury (Do et al, Gastroenterol. Res. Pract. (2011), doi: 10.1155/2011/917941).

As stated above, inflammation is a key part of a normal immune response in that it leads to healing at a local level. Radiation proctitis on the other hand is a condition that is characterised by injury, damage and wounding, but in which a patient's immune response is suppressed, and so a localised inflammatory response does not tend to lead to the normal healing process, and can persist for extended periods. Further, local antinflammatory drugs such as corticosteroids have the tendency of making things worse by suppressing the immune response further.

Hence there is a clear unmet clinical need for an effective radiation proctitis treatment using an agent that is either directly capable of restoring the systemic immune response, is capable of treating the symptoms of radiation proctitis, as described hereinafter, without further compromising a patient's systemic immune response, or is capable of doing both of these things, that is reducing those symptoms whilst working in a manner that either has immunorestorative effect or, at the very least, does not have an immunosuppressive effect.

Montelukast is an orally active non-steroidal immunomodulating compound that is administered perorally to the gastrointestinal tract for the maintenance treatment and prevention of symptoms of seasonal allergies (see e.g. Hon et al, Drug Design, Development and Therapy, 8, 839 (2014)). It acts by blocking the action of, primarily, leukotriene D4 (as well as leukotrienes C4 and E4) on the cysteinal leukotriene receptor CysLT1 in the airways. It is known to treat chronic, allergic conditions, like asthma, with a low dose (4 mg to 10 mg daily) in tablet form.

In this respect, in a similar manner to steroids, montelukast is typically administered in a manner in which it treats the consequences of an over-active immune system, in which a subject's immune system is reacting to otherwise harmless allergens. As described in the review article by Theron et al (J. Immunol. Res., http://dx.doi.org/10.1155/2014/608930), the action of montelukast on CysLTs serves to reduce the severity of inflammatory response that is a part of innate immune system.

In international patent application WO 2019/007356, it is described how topical compositions comprising montelukast were unexpectedly found to promote recovery of open wounds and burns when applied directly thereto.

As described hereinafter, we have surprisingly found that montelukast is capable of restoring radiation-induced suppression of the immune response in an animal model, and thus has unexpected immunorestorative properties. This renders it of potential use in the treatment of immunosuppressive disorders and/or in the treatment of disorders or symptoms thereof, including wounding, induced in patients with compromised immune systems, as is the case in radiation proctitis.

### DISCLOSURE OF THE INVENTION

In this respect, and according to an aspect of the invention, there is provided the use of montelukast or a pharmaceutically acceptable salt thereof for the manufacture of a medicament for the treatment of a condition characterized by immunosuppression in a patient.

According to a further aspect of the invention, there is provided the use of montelukast or a pharmaceutically acceptable salt thereof for the manufacture of a medicament for the treatment of an immunodeficiency disorder, for the treatment of a patient with a compromised immune system, as well as for the restoration of the normal function of the immune system of a patient.

Montelukast may be presented in the form of a salt. Salts that may be mentioned include pharmaceutically acceptable salts, such as pharmaceutically acceptable acid addition salts and base addition salts. Such salts may be formed by conventional means, for example by reaction of montelukast with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of active ingredient in the form of a salt with another counter-ion, for example using a suitable ion exchange resin.

Preferred salts include, for example, acetate, hydrochloride, bisulfate, maleate, mesylate, tosylate, alkaline earth metal salts, such as calcium and magnesium, or alkali metal salts, such as the potassium salt and particularly the sodium salt.

Conditions characterized by immunosuppression (or immunodeficiency disorders) include PIDDs, and thus include humoral immunodeficiency disorders, such as common variable immunodeficiency, selective immunoglobulin deficiency (e.g. IgA deficiency), transient hypogammaglobulinemia of infancy, X-linked agammaglobulinemia; cellular immunodeficiency disorders, such as chronic mucocutaneous candidiasis, DiGeorge syndrome, X-linked lymphoproliferative syndrome; combined humoral and cellular immunodeficiency disorders, such as ataxia-telangiectasia, hyperimmunoglobulinemia E syndrome, severe combined immunodeficiency, Wiskott-Aldrich syndrome; phagocytic immunodeficiencies, such as Chediak-Higashi syndrome, chronic granulomatous disease, cyclic neutropenia, leukocyte adhesion defects; and complement deficiencies, such as complement component 1 (C1) inhibitor deficiency (or hereditary angioedema), C3 deficiency, C4 deficiency, as well as C5, C6, C7, C8, and/or C9 deficiencies.

However, we prefer that the immunodeficiency disorders that is treated in accordance with the invention is a SIDD, that is an immunodeficiency disorder that is caused by a secondary factor, such as old age, malnutrition (e.g. undernutrition), a chronic disorder, one or more chemical agents (e.g. drugs) and/or (e.g. ionizing) radiation.

Disorders that can cause immunodeficiency in patients include cancers; disorders of the blood, such as aplastic anaemia, leukaemia, multiple myeloma; sickle cell disease; Down's syndrome; infections such as viral infections, including varicella, cytomegalovirus, Epstein-Barr virus, HIV, measles and bacterial infections; diabetes mellitus; diseases of internal organs, such as chronic kidney disease, nephrotic syndrome, chronic hepatitis, liver failure; systemic lupus erythematosus; alcoholism, chronic burns; and operations, such as removal of the spleen.

Drugs that can cause immunodeficiency in patients include antiseizure drugs, such as lamotrigine, phenytoin, valproate; immunosuppressants, such as azathioprine, cyclosporine, everolimus, leflunomide, mycophenolate, mofetil, sirolimus, tacrolimus, tofacitinib; biologics, such as abatacept, adalimumab, anakinra, basiliximab, certolizumab, daclizumab, etanercept, golimumab, infliximab, ixekizumab, muromonab (OKT3), natalizumab, rituximab, secukinumab, tocilizumab, ustekinumab, vedolizumab; and, particularly, corticosteroids, such as naturally occurring corticosteroids, including cortisol (hydrocortisone), aldosterone, corticosterone, cortisone, pregnenolone, progesterone, as well as naturally occurring precursors and intermediates in corticosteroid biosynthesis, and other derivatives of naturally occurring corticosteroids, such as 11-deoxycortisol, 21-deoxycortisol, 11-dehydrocorticosterone, 11-deoxycorticosterone, 18-hydroxy-11-deoxycorticosterone, 18-hydroxycorticosterone, 21-deoxycortisone, 11β-hydroxypregnenolone, 11β,17α,21-trihydroxypregnenolone, 17α,21-dihydroxypregnenolone, 17α-hydroxypregnenolone, 21-hydroxypregnenolone, 11-ketoprogesterone, 11β-hydroxyprogesterone, 17α-hydroxyprogesterone and 18-hydroxyprogesterone, and synthetic corticosteroids, including those of the hydrocortisone-type (Group A), such as cortisone acetate, hydrocortisone aceponate, hydrocortisone acetate, hydrocortisone buteprate, hydrocortisone butyrate, hydrocortisone valerate, tixocortol and tixocortol pivalate, prednisolone, methylprednisolone, prednisone, chloroprednisone, cloprednol, difluprednate, fludrocortisone, fluocinolone, fluperolone, fluprednisolone, loteprednol, prednicarbate and triamcinolone; acetonides and related substances (Group B), such as amcinonide, budesonide, desonide, fluocinolone cetonide, fluocinonide, halcinonide, triamcinolone acetonide, ciclesonide, deflazacort, formocortal, fludroxycortide, flunisolide and fluocinolone acetonide, those of the (beta)methasone-type (Group C), such as beclomethasone, betamethasone, betamethasone dipropionate and valerate, dexamethasone, fluocortolone, halometasone, mometasone and mometasone furoate, alclometasone and alclometasone dipropionate, clobetasol and clobetasol propionate, clobetasone and clobetasone butyrate, clocortolone, desoximetasone, diflorasone, difluocortolone, fluclorolone, flumetasone, fluocortin, fluprednidene and fluprednidene acetate, fluticasone, fluticasone furoate and fluticasone propionate, meprednisone, paramethasone, prednylidene, rimexolone and ulobetasol; those of the progesterone-type, such as flugestone, fluorometholone, medrysone and prebediolone acetate, and progesterone derivatives (progestins), such as chlormadinone acetate, cyproterone acetate, medrogestone, medroxyprogesterone acetate, megestrol acetate and segesterone acetate; as well as other corticosteroids, such as cortivazol and 6-methyl-11β,17β-dihydroxy-17α-(1-propynyl)androsta-1,4,6-trien-3-one. Particular corticosteroids that may be mentioned include cortisone, prednisone, prednisolone, methylprednisolone and dexamethasone.

Drugs that may cause immunodeficiency in patients that may particularly be mentioned however include chemotherapeutic treatments of cancers, such as alemtuzumab, busulfan, cyclophosphamide, melphalan.

Particular SIDDs that may be mentioned include those caused by radiation therapy that is employed to treat disorders such as cancer (i.e. radiation-induced immunosuppression).

Ionizing radiation not only suppresses the immune system in the manner described hereinbefore, but also can alter the functions of the immune system in irradiated organs in other ways. For example, increased levels in inflammatory mediators, such as NF-κB and SMAD2/3, and cytokines, such as IL-1, IL-2, IL-6, IL-8, IL-33, tumor necrosis factor (TNF-α), transforming growth factor beta (TGF-β) and interferon gamma (IFN-γ) are associated with the release of prostaglandins and free radicals, including reactive oxygen species (ROS) and nitric oxide (NO). Exposure to high doses of radiation that may occur during accidental exposure (e.g. as a consequence of a nuclear or a radiologic disaster) may result in inflammatory responses and/or wounding, which may continue for years afterwards and/or disrupt the functions of irradiated organs.

Thus, although, as described herein, montelukast has unexpectedly been found to be capable of treating radiation-induced immunosuppression per se, because it is also known to possess both antiinflammatory and wound-healing properties, particularly when administered topically to the site of inflammation and/or wounding, its immunorestorative properties mean that it is particularly useful in the treatment of conditions characterised by inflammation and/or wounding in a patient with a compromised immune system. Such patients include those having one or more of the aforementioned conditions characterised by immunodeficiency, and in particular include those with radiation-induced inflammation, wounding and/or immunosuppression.

In particular, in the treatment of such radiation-induced conditions, montelukast and salts thereof may be employed not only to provide an immunorestorative effect, but also to simultaneously promote wound recovery and/or healing. This is particularly useful in view of the fact that wounds that are associated with such a condition are difficult, if not impossible, to treat properly in view of the immunosuppressive effect induced by the radiation and the absence of a normal endogenous inflammatory response.

Furthermore, by providing the aforesaid immunorestorative effect, this enables the body's immune system and localised inflammatory response to become more effective and, in this respect, montelukast and salts thereof may also be employed to provide an antiinflammatory effect, at the same time as promoting further wound healing, but in a manner that does not compromise further the patient's immune system (in the manner that a corticosteriod would if it were employed to treat the inflammation).

According to a further aspect of the invention, there is provided the use of montelukast or a pharmaceutically acceptable salt thereof for the manufacture of a medicament for the treatment of inflammation and/or of a condition characterized by inflammation or wounding, in a patient that has, or is vulnerable to, a condition characterized by immunosuppression, which includes the treatment of radiation-induced conditions characterized by inflammation and/or wounds.

Diseases (including those characterised by inflammation and/or wounds) that are radiation-induced per se, and/or may result from radiation-induced immunosuppression, include those that may arise following accidental exposure to radiation (commonly known as ‘radiation poisoning’), or following deliberate and/or targeted exposure to radiation, for example as a consequence of (e.g. ionizing) radiation therapy to treat a disease, such as cancer.

Radiation therapy is a type of e.g. cancer treatment that uses an external beam of intense energy to kill cancer cells. Radiation therapy most often uses X-rays, but protons or other types of energy also can be used. Radiation therapy may be used as a primary cancer treatment, in neoadjuvant therapy (shrinking a cancerous tumor before surgery), adjuvant therapy (preventing proliferation of cancer cells after surgery), to alleviate symptoms caused by advanced cancer, or two or more of the above in combination. Radiation therapy may also be used in combination with other treatments, such as chemotherapy.

Disorders characterised by inflammation and/or wounding of the mucosa and/or skin that may result from exposure to radiation are often associated with the part of the body that is targeted/irradiated. For example:
radiation-induced dermatitis and mucositis may occur in the skin or the mucosa, respectively, at locations that may be close to the part of the body that is irradiated. For example, radiation-induced oral mucositis may occur following irradiation of the head or neck;
radiation-induced encephalitis may also occur following irradiation of the head or neck; and
radiation pneumonitis and/or radiation esophagitis often result from radiation treatment of lung cancers, breast cancer, lymphomas, thymic tumours, or oesophageal cancer with radiation.

Radiation treatment that is aimed at the abdomen, pelvis or rectum (e.g. to treat cancer of the cervix, the prostate, the bladder or the rectum) may result in one or more of radiation enteropathy (or radiation enteritis, including radiation colitis), radiation hepatitis, radiation myelitis, radiation vaginitis and, particularly, radiation proctitis.

In particular, radiation proctitis or radiation proctopathy is condition characterized by damage to the rectum after exposure to radiation during radiation therapy. Inflammation can be acute (acute radiation proctitis, as well as the related radiation colitis), or chronic (e.g. radiation associated vascular ectasias (RAVE) and chronic radiation proctopathy).

Initial symptoms of acute radiation proctitis include pelvic pain, diarrhoea and tenesmus but radiation damage to the rectum often causes incontinence and rectal bleeding, with severe cases leading to wounds, strictures and/or fistulae.

Thus, in the treatment of disorders induced by irradiation for e.g. cancer therapy, more particularly irradiation of the lower abdominal region, including disorders such as radiation proctitis as defined above, radiation colitis and radiation-induced dermatitis, montelukast and salts thereof may be employed:
to provide an immunorestorative effect at the same time as treating wounds and/or promoting recovery and/or healing of wounds that are associated with such a disorder; and/or
to provide a more direct antiinflammatory effect without compromising the patient's immune system further, at the same time as being available to promote further wound healing.

According to two further aspects of the invention there are provided:
a method of treatment of a radiation-induced condition that is characterized by (i) immunosuppression, and (ii) inflammation and/or wounding; and
a method of treating inflammation and/or wounds that are associated with a radiation-induced immunodeficiency disorder whilst simultaneously restoring the normal function of the immune system of a patient, which methods comprise administering montelukast or a pharmaceutically acceptable salt thereof to a patient in need thereof.

The methods of treatment and uses described herein are particularly useful when the disorder that is induced by irradiation for e.g. cancer therapy, result from irradiation of the lower abdominal region as described above.

According to a still further aspect of the invention there is provided a method of reduction in the incidence of morbidity and/or mortality that is or may be associated with of radiation- (e.g. ionizing radiation-)induced disorder characterised by inflammation and/or wounding in a patient, which method comprises administration of montelukast, or a pharmaceutically acceptable salt thereof to a patient in need of such treatment.

For the avoidance of doubt, in the context of the present invention, the terms ‘treatment’, ‘therapy’ and ‘therapy method’ include the therapeutic, or palliative, treatment of patients in need of, as well as the prophylactic treatment and/or diagnosis of patients which are susceptible to the aforementioned disorders or conditions.

Whether a treatment has restored the normal function of the immune system of a patient may be determined either by objective measures (e.g. biomarkers, such as those described hereinafter), or by subjective measures (e.g. in the patient's own opinion or, more likely, that of a qualified medical practitioner). The term will also be understood to include not only the full restoration of a patient's immune response to normal levels, but also its partial-restoration, and even not deteriorating to an expected degree over time, for example as compared to baseline levels and/or in accordance with normal/expected progression of deterioation, during the course of the immunosuppression and/or immunodeficiency disorder.

‘Patients’ include reptilian, avian and, preferably, mammalian (particularly human) patients. In this respect, the terms ‘pharmaceutical’ and ‘pharmaceutically acceptable’ include ‘veterinary’ and ‘veterinarily-acceptable’.

In accordance with the invention, montelukast and pharmaceutically acceptable salts thereof many administered locally or systemically, for example orally, intravenously or intraarterially (including by intravascular and other perivascular devices/dosage forms (e.g. stents)), intramuscularly, cutaneously, subcutaneously, transmucosally (e.g. sublingually or buccally), intramucosally, rectally or intrarectally, intravaginally, intradermally, transdermally, nasally, pulmonarily (e.g. tracheally or bronchially), preferably topically, by way of direct injection or by any other parenteral route, in the form of a pharmaceutical preparation comprising montelukast or salt thereof in a pharmaceutically acceptable dosage form(s).

Direct systemic administration may be achieved by way of normal peroal administration and absorption of active ingredient through the gastrointestinal tract, or by direct parenteral administration, for example transdermally or transmucosally (for example absorption of active ingredient through any mucous membrane (including the rectum, the vagina, the nasal cavity, the oral cavity or the gastrointestinal tract, including the lower intestine, such as the colon and/or the anorectal mucosa), or by way of intradermal and/or intramucosal injection to the same biological surfaces.

Montelukast or salt thereof may in the alternative be administered by direct local and or topical administration. For example, injection may be local (e.g. intradermal, intramucosal or subcutaneous) to a relevant tissue, such as the spinal column (an epidural), or locally, with a view to producing a systemic effect, for example by direct injection in the bone marrow.

Local, topical (and particularly mucosal) administration of montelukast may give rise not only to a local effect, but also to a systemic effect (as a consequence of systemic absorption, as stated above).

Whether for local/topical or systemic administration, pharmaceutically acceptable formulations for use in injection may comprise monteukast or pharmaceutically acceptable salt thereof in admixture with a pharmaceutically acceptable adjuvant, diluent or carrier, which may be selected with due regard to the intended route of direct parenteral administration and standard pharmaceutical practice. Such pharmaceutically acceptable carriers may be chemically inert to the active compound and may have no detrimental side effects or toxicity under the conditions of use. Such pharmaceutically acceptable carriers may also impart an immediate, or a modified, release of monteukast or pharmaceutically acceptable salt thereof.

Formulations for injection may thus be in the form of an aqueous formulation such as an a suspension and/or, more preferably a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution), or a freeze-dried powder that may be reconstituted with a vehicle, such as an aqueous vehicle prior to use (e.g. injection)).

Formulations for injection may include other suitable excipients known to those skilled in the art, such as solvents (e.g. water), co-solvents, solubilizing agents (e.g. cyclodextrins), wetting agents, suspending agents, emulsifying agents, thickening agents, chelating agents, antioxidants, reducing agents, antimicrobial preservatives, bulking agents and/or protectants.

Formulations for injection are preferably buffered by standard techniques to physiologically acceptable pH values (e.g. pHs of between about 4.5 and about 9.5, e.g. about 6 and about 9, such as between about 6.5 and about 8.5) using buffers and/or pH modifiers as described herein, and/or may further comprise tonicity-modifying agents (such as sodium chloride).

In addition, montelukast or salt thereof may be administered in a targeted fashion, in which known galenic manipulations are employed to target delivery of a composition, following parenteral or peroal administration, to one or more internal organs of a patient, such as the stomach, the intestines, the pancreas, the liver, the spleen, the bladder, the kidneys, the lungs, the cardiovascular system (including the heart and the vascular system), the ovaries, the prostate, the central nervous system, the bone marrow, the eyes, the vagina, the cervix, etc.

For example, administration by targeted, local delivery to the lower gastrointestinal tract may be achieved by parenteral, and particularly by peroral, delivery, by means of standard delayed- or extended-release coating techniques known to those skilled in the art. In particular, distinct parts of the upper or lower intestine may be targeted. For example, colonic administration can also be achieved by way of colon-targeted drug delivery means that are initially administered perorally or parenterally.

Topical administration may also be achieved by way of inhalation, for example intranasally or pulmonarily to the lung. Topical formulations may be administration this way by creating a spray comprising montelukast or salt thereof, e.g. by using a powder aerosol or by way of an aqueous mist using an appropriate atomization technique or apparatus, such as a nebulizer.

Local means of delivery of monteukast or pharmaceutically acceptable salts thereof also include direct topical application (e.g. to the mucosa, including the oral and/or nasal mucosae, the lung, the anorectal area and/or the colon, or to the skin) in an appropriate (for example pharmaceutically- and topically acceptable) vehicle suitable for application to the skin and/or the appropriate mucosal surface. Such vehicles may be commercially available, and may also be suitable for oral, intravenous, cutaneous or subcutaneous, nasal, intramuscular, intraperitoneal, or pulmonary delivery.

Topical formulations comprising monteukast or pharmaceutically acceptable salts thereof will generally be administered in the form of one or more pharmaceutical formulations in admixture with a (e.g. pharmaceutically- and/or topically acceptable) adjuvant, diluent or carrier, which may be selected with due regard to the intended route of administration (e.g. topical to the relevant mucosa (including the lung) or, preferably, the skin) and standard pharmaceutical or other (e.g. cosmetic) practice. Such pharmaceutically acceptable carriers may be chemically inert to the active compounds and may have no detrimental side effects or toxicity under the conditions of use. Such pharmaceutically acceptable carriers may also impart an immediate, or a modified, release of montelukast.

Suitable pharmaceutical formulations may be commercially available or otherwise prepared according to techniques that are described in the literature, for example, Remington The Science and Practice of Pharmacy, 22^{nd} edition, Pharmaceutical Press (2012) and Martindale—The Complete Drug Reference, 38^{th} Edition, Pharmaceutical Press (2014) and the documents referred to therein, the relevant disclosures in all of which documents are hereby incorporated by reference. Otherwise, the preparation of suitable formulations including montelukast and salts thereof may be achieved non-inventively by the skilled person using routine techniques.

Montelukast and salts thereof may further and/or in the alternative be combined with appropriate excipients to prepare:
gel formulations (for which suitable gel matrix materials include cellulose derivatives, carbomer and alginates, gummi tragacanthae, gelatin, pectin, carrageenan, gellan gum, starch, Xanthan gum, cationic guar gum, agar, noncellulosic polysaccharides, saccharides such as glucose, glycerin, propanediol, vinyl polymers, acrylic resins, polyvinyl alcohol, carboxyvinyl polymer and, particularly, hyaluronic acid);
lotions (for which suitable matrix materials include cellulose derivatives, glycerin, noncellulosic polysaccharides, polyethylene glycols of different molecular weights and propanediol);
pastes or ointments (for which suitable paste matrix materials include glycerin, vaseline, paraffin, polyethylene glycols of different molecular weights, etc.);
creams or foams (for which suitable excipients (e.g. foaming agents) include hydroxypropyl methyl cellulose, gelatin, polyethylene glycols of different molecular weights, sodium dodecyl sulfate, sodium fatty alcohol polyoxyethylene ether sulfonate, corn gluten powder and acrylamide);
powder aerosols (for which suitable excipients include mannitol, glycine, dextrin, dextrose, sucrose, lactose, sorbitol and polysorbates, e.g. a dry powder inhalant);
liquid, for example, water (aerosol) sprays for oral use or for inhalation (for which suitable excipients include viscosity modifiers, such as hyaluronic acid, sugars, such as glucose and lactose, emulsifiers, buffering agents, alcohols, water, preservatives, sweeteners, flavours, etc.); and/or
injectable solutions or suspensions (which may be aqueous or otherwise and for which suitable excipients include solvents and co-solvents, solubilizing agents, wetting agents, suspending agents, emulsifying agents, thickening agents, chelating agents, antioxidants, reducing agents, antimicrobial preservatives, buffers and/or pH modifiers, bulking agents, protectants and tonicity-modifying agents), particular injectable solutions or suspensions that may be mentioned include dermal fillers (i.e. injectable fillers or soft-tissue fillers), particularly when the c montelukast/salt thereof is combined with hyaluronic acid.

Moisturizing agents, such as glycerol, glycerin, polyethylene glycol, trehalose, glycerol, petrolatum, paraffin oil, silicone oil, hyaluronic acid and salts (e.g. sodium and potassium salts) thereof, octanoic/caprylic triglyceride, and the like; and/or antioxidants, such as vitamins and glutathione; and/or pH modifiers, such as acids, bases and pH buffers, may also be included in such formulations, as appropriate.

Furthermore, surfactants/emulsifiers, such as hexadecanol (cetyl alcohol), fatty acids (e.g. stearic acid), sodium dodecyl sulfate (sodium lauryl sulfate), sorbitan esters (e.g. sorbitan stearate, sorbitan oleate, etc.), monoacyl glycerides (such as glyceryl monostearate), polyethoxylated alcohols, polyvinyl alcohols, polyol esters, polyoxyethylene alkyl ethers (e.g. polyoxyethylene sorbitan monooleate), polyoxyethylene castor oil derivatives, ethoxylated fatty acid esters, polyoxylglycerides, lauryl dimethyl amine oxide, bile salts (e.g. sodium deoxycholate, sodium cholate), lipids (e.g. fatty acids, glycerolipids, glycerophospholipids, sphingolipids, sterols, prenols, saccharolipids, polyketides), phospholipids, N,N-dimethyldodecylamine-N-oxide, hexadecyltrimethyl-ammonium bromide, poloxamers, lecithin, sterols (e.g. cholesterol), sugar esters, polysorbates, and the like; preservatives, such as phenoxyethanol, ethylhexyl glycerin, and the like; and thickeners, such as acryloyldimethyltaurate/VP copolymer, may be included. In particular, stearic acid, glyceryl monostearate, hexadecanol, sorbitan stearate, cetyl alcohol, octanoic/capric glyceride etc. may be included, particularly in cream formulations.

Montelukast and salts thereof, and (e.g. pharmaceutical) formulations (e.g. aqueous solutions, gels, creams, ointments, lotions, foams, pastes and/or dry powders as described above) including them, may further be combined with an appropriate matrix material to prepare a dressing or a therapeutic patch for application on a biological surface, such as the skin or a mucosal surface. Such formulations may thus be employed to impregnate a matrix material, such as gauze, non-woven cloth or silk paper. The therapeutic patch may alternatively be, for example, a band-aid, a facial mask, an eye mask, a hand mask, a foot mask, etc.

Vaseline may be employed for use in applying such dressings to wounds, but we have also found that ointments based on PEGs (e.g. PEG 400) may be combined with matrix materials to prepare dressings without the need to use Vaseline.

Montelukast and salts thereof may also be used in combination with solid supports (such as nasal dressings (for example, to stop nasal bleeding), dermal scaffolds (for example, in wound healing) or artificial bones (for example, in the case of bone grafting/implantation).

Gels for topical administration (e.g. to mucosal surfaces as described herein) may comprise excipients such as solubilizing agents (e.g. dextrins, such as cyclodextrins including hydroxypropyl-beta-cyclodextrin), thickening or suspending agents (e.g. hydroxypropyl methylcellulose, gelatin, polyethylene glycols, etc.) chelating agents (such as sodium edetate) antimicrobial preservatives, buffers and/or pH modifiers, in addition to water.

Montelukast and salts thereof may be administered for inhalation by way of suspension, a dry powder or a solution. Suitable inhalation devices include pressurized metered-dose inhalers (pMDIs), which may be hand- or breath-actuated and employed with or without a standard spacer device, dry powder inhalers (DPIs), which may be single-dose, multi-dose, and power-assisted, and soft mist inhalers (SMIs) or nebulizers, in which aerosol drug in a fine mist is delivered with slower velocity than a spray delivered using, for example, a pMDI.

In pMDIs, montelukast and salts thereof may be administered as a pressurized suspension of micronized particles distributed in a propellant (e.g. HFA, along with excipients, such as mannitol, lactose, sorbitol, etc.), or as an ethanolic solutions, to deliver one or more metered dose of between about 20 and about 100 μL with each actuation. Actuation may be effected by hand (e.g. pressing) or by inhalation (breath-actuation), involving a flow-triggered system driven by a spring.

In DPIs, montelukast and salts thereof may be administered in the form of micronized drug particles (of a size between about 1 and about 5 μm), either alone or blended with inactive excipient of larger particle size (e.g. mannitol), inside a capsule, which may be pre-loaded or manually loaded into the device. Inhalation from a DPI may de-aggregate the medication particles and disperse them within the airways.

In SMIs, montelukast and salts thereof may be stored as a solution inside a cartridge, which is loaded into the device. A spring may release the dose into a micropump, such that the dose is released when a button is pressed, releasing jet streams of drug solution.

Various nebulizers may also be used to administer montelukast and salts thereof in the form of a fine mist of aerosolized solution. Nebulizers may include breath-enhanced jet nebulizer (in which, with the assistance of a compressor, an air stream moves through jet causing drug solution to be aerosolized); breath-actuated jet nebulizers (in which, after a patient inhales, with the assistance of a compressor, an air stream moves through a tube causing the drug solution to be aerosolized); ultrasonic nebulizers (in which piezoelectric crystals vibrate causing aerosolization by heating causing nebulization); vibrating mesh nebulizers (in which piezoelectric crystals vibrate a mesh plate causing aerosolization to give very fine droplets without a significant change in temperature of the solution during nebulization).

However, when the condition to be treated is radiation proctitis, topical, anorectal administration is particularly useful, using an appropriate delivery means, such as indirect topical administration of one or more of the targeted/delayed released compositions described hereinbefore, or by direct topical administration of a solution, a foam or a gel to be applied manually and/or as an enema (e.g. a foam enema, a gel enema or a liquid enema), by intrarectal injection, or by way of a suppository.

Compositions comprising montelukast for use in accordance with the invention may be (or may preferably be) sterile, or sterilized, prior to administration, in order to meet appropriate regulatory standards. Sterilization may be carried out by an in situ sterilization processes, such as sterilizing filtration and/or aseptic processing, or by a terminal sterilization process, such as through heat, which includes dry heat sterilization and moist heat sterilization (e.g. in an autoclave).

According to a further aspect of the invention there is provided a (e.g. pharmaceutical) composition comprising montelukast, or a pharmaceutically acceptable salt thereof and one or more pharmaceutically acceptable excipient, such as an adjuvant, diluent or carrier for use in the above-mentioned conditions.

Preferred pharmaceutical compositions comprising montelukast, or a pharmaceutically acceptable salt thereof that are suitable for, adapted for, and/or packaged and presented for topical administration (e.g. to the mucosa, including the skin, the oral and/or nasal mucosa, the lung, the colon, and/or, particularly, the anorectal area), for use in the treatment of a immunosuppressive disorder, or the treatment of wounding, inflammation or a condition characterized by inflammation in a patient with a suppressed immune system, which suppressed immune system may be brought on by radiation therapy for e.g. cancer, by way of direct topical administration of that formulation (e.g. to the skin, to the mucosa, including the oral and/or nasal mucosa, the lung, the colon and/or, particularly, the anorectal area), and/or by intradermal, subcutaneous and/or intramucosal injection.

For the avoidance of doubt, topical formulations comprising montelukast or salts thereof may be used in any and all conditions described herein, including treatments of any immunosuppressive disorder described herein, or inflammation in a patient with a suppressed immune system, which may, for example, be brought on by radiation therapy for e.g. cancer, as hereinbefore mentioned, defined or described. Similarly, topical formulations comprising montelukast or salts thereof that may be mentioned include any and all of those mentioned, defined or described herein. Any and all of the relevant disclosures herein are hereby incorporated by reference in conjunction with this aspect of the invention.

Administration of the montelukast or salts thereof may be continuous or intermittent. The mode of administration may also be determined by the timing and frequency of administration, but is also dependent, in the case of therapeutic treatment, on the severity of the condition.

Depending on the disorder, and the patient, to be treated, as well as the route of administration, montelukast or salts thereof may be administered at varying therapeutically effective doses to a patient in need thereof.

Similarly, the amount of the montelukast or salts thereof in a formulation will depend on the severity of the condition, and on the patient, to be treated, but may be determined by the skilled person.

In any event, the medical practitioner, or other skilled person, will be able to determine routinely the actual dosage, which will be most suitable for an individual patient, depending on the severity of the condition and route of administration. The dosages mentioned herein are exemplary of the average case; there can, of course, be individual instances where higher or lower dosage ranges are merited, and such are within the scope of this invention.

Doses may be administered between once and four (e.g. three) times daily.

Appropriate concentrations of montelukast or salts thereof in an aqueous solution product may be about 0.01 (e.g. about 0.1) to about 15.0 mg/ml, in all cases calculated as free montelukast.

Appropriate topical (including topically applied) doses of montelukast or salts thereof are in the range of about 0.05 to about 50 (e.g. about 20) μg/cm² of treated area, such as about 0.1 (e.g. about 0.5) to about 20 (e.g. about 5) μg/cm² of treated area, including about 1 to about 10 μg/cm² of treated area, such as about 5 μg/cm² of treated area, in all cases calculated as free montelukast.

In any event, the dose administered to a mammal, particularly a human, in the context of the present invention should be sufficient to affect a therapeutic response in the mammal over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

Montelukast or salts thereof may be combined with a multitude of known pharmaceutically active ingredients, for use in the treatment of an immunosuppressive disorder and/or in the treatment of inflammation, or of a condition characterized by inflammation, in a patient with a compromised immune system, including any therapeutic agent, or drug, that is:
capable of producing some sort of physiological effect (whether in a therapeutic or prophylactic capacity against a particular disease state or condition) in a living subject, including, in particular, mammalian and especially human subjects (patients), and which will work ‘in concert’ with montelukast or salt thereof to treat the conditions to be treated; or
is known or suspected to cause immunosuppression and/or a combination of inflammation and immunosuppression, which can be counteracted by the immunorestorative and/or antiinflammatory properties of montelukast.

In relation to pharmaceutically active/therapeutic agents that are known or suspected to cause immunosuppression and/or a combination of inflammation and immunosuppression, these include any one or more of those described hereinbefore, which are incorporated into this aspect of the invention by reference.

Pharmaceutically active agents may have an effect that is cumulative, additive and/or synergistic to the immunorestorative and/or antiinflammatory properties of montelukast may be selected from certain other anti-inflammatory agents, antibiotics, anti-bacterial and/or antiprotozoal agents, antiviral agents (e.g. protease inhibitors).

Anti-inflammatory drugs that may be used in accordance with the uses and methods of treatment described herein include those used in the treatment of autoimmune diseases, such arthritis (such as cataflam, betamethasone, naproxen, cyclosporin, chondroitin, celecoxib, etodolac, meclofenamate, salsalate, methylprednisolone, and piroxicam); and osteoarthritis (such as sulindac, meloxicam, fenoprofen, etoricoxib, and nabumetone).

Non-limiting examples of anti-bacterial drugs which may be used in accordance with the uses and methods of treatment described herein include chloramphenicol, ofloxacin, levofloxacin, tobramycin, norfloxacin, ciprofloxacin, lomefloxacin, lincomycin, fluconazole, enoxacin, furazolidone, nitrofurazone, rifampicin, micronomicin, gentamicin, cetylpyridinium, neomycin, roxithromycin, sulfadiazine silver, clarithromycin, clindamycin, metronidazole, azithromycin, mafenide, sulfamethoxazole, paracetamol, chloramphenicol, pseudoephedrine, mupirocin, amoxicillin, amoxicillin/clavulanic acid, trimethoprim/sulfamethoxazole, cefalexin, moxifloxacin, known or commercially available pharmaceutically acceptable salts of any of the foregoing, and combinations of any of the foregoing compounds and/or salts.

Non-limiting examples of antiviral drugs which may be used in accordance with the uses and methods of treatment described herein include tobramycin ribavirin, acyclovir, moroxydine, foscarnet, ganciclovir, idoxuridine, trifluridine, brivudine, vidarabine, entecavir, telbivudine, foscarnet, zidovudine, didanosine, zalcitabine, stavudine, lamivudine, abacavir, emtricitabine, nevirapine, delavirdine, efavirenz, etravirine, rilpivirine, saquinavir, ritonavir, indinavir, nelfinavir, amprenavir, lopinavir, ritonavir, atazanavir, fosamprenavir, tipranavir, darunavir, telaprevir, boceprevir, simeprevir, asunaprevir, raltegravir, elvitegravir, dolutegravir, rsv-igiv, palivizumab, docosanol, enfuvirtide, maraviroc, vzig, varizig, acyclovir, ganciclovir, famciclovir, valacyclovir, penciclovir, valganciclovir, cidofovir, tenofovir disoproxil fumarate, adefovir dipivoxil, fomivirsen, podofilox, imiquimod, sinecatechins, interferon-α 2b (recombinant, human), known or commercially available pharmaceutically acceptable salts of any of the foregoing, and combinations of any of the foregoing compounds and/or salts.

Montelukast and salts thereof may also be combined in accordance with the uses and methods of treatment described herein with stem cells (e.g. totipotent (omnipotent), pluripotent (such as embryonic or induced pluripotent stem cells), multipotent (such as mesenchymal stem cells), oligopotent (such as hematopoietic stem cells), or unipotent (such as muscle stem cells)).

Patients may also (and/or may already) be receiving therapy based upon administration of one or more of the aforementioned other, known pharmaceutically active ingredients, for example to treat one or more of the conditions described herein, by which we mean receiving a prescribed dose of one or more of the active ingredients mentioned herein, prior to, in addition to, and/or following, treatment with montelukast or salt thereof.

Such other pharmaceutically active ingredients may also be administered in combination with montelukast or salts thereof in numerous ways.

For example, montelukast and salts thereof may be ‘combined’ with the other pharmaceutically active ingredients (or ‘therapeutic agents’) for administration together in the same (e.g. pharmaceutical) formulation, or administration separately (simultaneously or sequentially) in different (e.g. pharmaceutical) formulations.

Thus, such combination products provide for the administration of montelukast or salts thereof in conjunction with the other therapeutic agent, and may thus be presented either as separate formulations, wherein at least one of those formulations comprises a montelukast/salt thereof, and at least one comprises the other therapeutic agent, or may be presented (i.e. formulated) as a combined preparation (i.e. presented as a single formulation including montelukast/salt thereof and the other therapeutic agent).

Thus, there is further provided:
(1) a (e.g. pharmaceutical) formulation including a montelukast or a pharmaceutically acceptable salt thereof; another pharmaceutically active ingredient as hereinbefore described; and a pharmaceutically acceptable inactive excipient (e.g. adjuvant, diluent or carrier), which formulation is hereinafter referred to as a ‘combined preparation’; and
(2) a kit of parts comprising components:
(A) montelukast or a pharmaceutically acceptable salt thereof in the form of a pharmaceutical formulation in admixture with a pharmaceutically acceptable inactive excipient (e.g. adjuvant, diluent or carrier); and
(B) another pharmaceutically active ingredient as hereinbefore described in the form of a pharmaceutical formulation in admixture with a pharmaceutically acceptable adjuvant, diluent or carrier,
which components (A) and (B) are each provided in a form that is suitable for administration in conjunction with the other.

In a further aspect of the invention, there is provided a process for the preparation of a combined preparation (1) as hereinbefore defined, which process comprises bringing into association montelukast/salt thereof, the other pharmaceutically active ingredient, and at least one pharmaceutically acceptable excipient.

In a further aspect of the invention, there is provided a process for the preparation of a kit-of-parts (2) as hereinbefore defined, which process comprises bringing into association components (A) and (B). As used herein, references to bringing into association will mean that the two components are rendered suitable for administration in conjunction with each other.

Thus, in relation to the process for the preparation of a kit-of-parts as hereinbefore defined, by bringing the two components ‘into association with’ each other, we include that the two components of the kit-of-parts may be:
(i) provided as separate formulations (i.e. independently of one another), which are subsequently brought together for use in conjunction with each other in combination therapy; or
(ii) packaged and presented together as separate components of a ‘combination pack’ for use in conjunction with each other in combination therapy.

Thus, there is further provided a kit of parts comprising:
(I) one of components (A) and (B) as defined herein; together with
(II) instructions to use that component in conjunction with the other of the two components.

The kits of parts described herein may comprise more than one (e.g. formulation including an) appropriate quantity/dose of montelukast/salt thereof, and/or more than one (e.g. formulation including an) appropriate quantity/dose of the other pharmaceutically active ingredient, in order to provide for repeat dosing. If more than one formulation comprising or quantity/dose of either of the foregoing is present, such may be the same, or may be different in terms of the dose of either compound, chemical composition(s) and/or physical form(s).

With respect to the kits of parts as described herein, by ‘administration in conjunction with’, we include that the respective components are administered, sequentially, separately and/or simultaneously, over the course of treatment of the relevant condition.

Thus, in respect of the combination product according to the invention, the term ‘administration in conjunction with’ includes that the two components of the combination product (montelukast/salt thereof and other pharmaceutically active ingredient) are administered (optionally repeatedly), either together, or sufficiently closely in time, to enable a beneficial effect for the patient, that is greater, over the course of the treatment of the relevant condition, than if either montelukast/salt thereof, or the other agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term ‘in conjunction with’ includes that one or other of the two components may be administered (optionally repeatedly) prior to, after, and/or at the same time as, administration of the other component. When used in this context, the terms ‘administered simultaneously’ and ‘administered at the same time as’ include that individual quantities/doses of montelukast/salt thereof and other active pharmaceutical ingredient are administered within 48 hours (e.g. 24 hours) of each other.

In addition, montelukast and pharmaceutically acceptable salts thereof may be provided in a form in which it is suitable for administration in conjunction with radiation therapy, that is administration of montelukast/salt thereof to a patient that is receiving, has received or will receive, irradiation therapy to treat a disease such as cancer.

By analogy with the foregoing, ‘administration of montelukast/salt in conjunction with radiation therapy’ includes that the two components (montelukast/salt thereof and the radiation therapy) are administered (optionally repeatedly), either together, or sufficiently closely in time, to enable a beneficial effect for the patient, that is greater, over the course of the treatment of the relevant condition, than if montelukast/salt thereof had not been administered (optionally repeatedly) over the same course of treatment (radiation therapy). Determination of whether this combination provides a greater beneficial effect in respect of, and over the course of treatment will depend upon the condition to be treated or prevented, but may be achieved routinely by the skilled person.

Further, in this context, the term ‘in conjunction with’ includes that montelukast/salt thereof is administered (optionally repeatedly) prior to, after, and/or at the same time as, administration of the radiation therapy. When used in this context, the terms ‘administered simultaneously’ and ‘administered at the same time as’ include that a quantity/dose of montelukast/salt thereof and the radiation therapy are administered up to about 60 days, or about 21 days, or about 10 days, or about 7 days, or within 48 hours (e.g. 24 hours) of each other.

Thus, in accordance with a further aspect of the invention, there is provided the use of montelukast or a pharmaceutically acceptable salt thereof for the manufacture of a medicament for the treatment of a radiation-induced inflammatory condition, such as radiation proctitis, which method comprises the administration of montelulkast or a pharmaceutically acceptable salt thereof to a patient that is receiving, has received or will receive, irradiation therapy to treat a disease such as cancer.

Wherever the word ‘about’ is employed herein, for example in the context of amounts, such as periods of times, concentrations and/or doses of active ingredients, particle sizes, volumes and pHs, it will be appreciated that such variables are approximate and as such may vary by ±10%, for example ±5% and preferably ±2% (e.g. ±1%) from the numbers specified herein. In this respect, the term ‘about 10%’ means e.g. ±10% about the number 10, i.e. between 9% and 11%.

The uses and methods described herein may also have the advantage that, in the treatment of the conditions mentioned hereinbefore, they may be more convenient for the physician and/or patient than, be more efficacious than, be less toxic than, have a broader range of activity than, be more potent than, produce fewer side effects than, or that it/they may have other useful pharmacological properties over, similar compounds or methods (treatments) known in the prior art, whether for use in the treatment of immunosuppressive disorders, and/or of inflammation in patients having disorders characterised by immunosuppression, or otherwise.

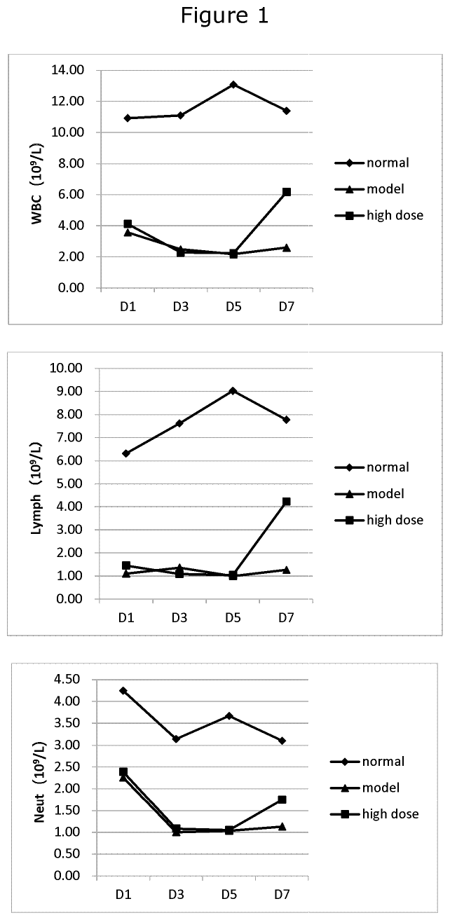

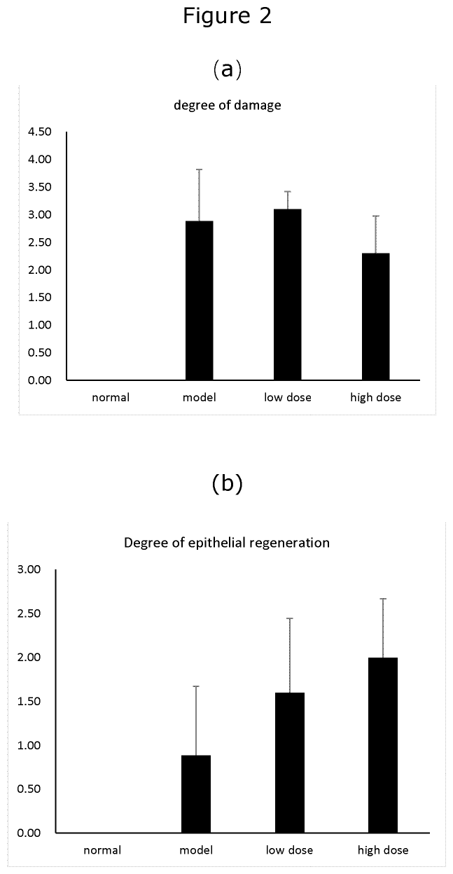

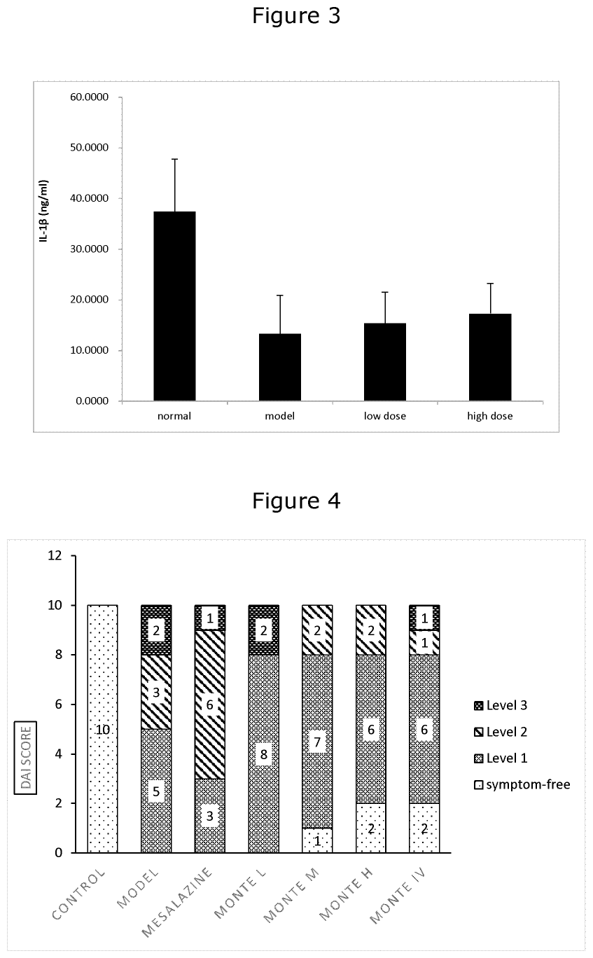

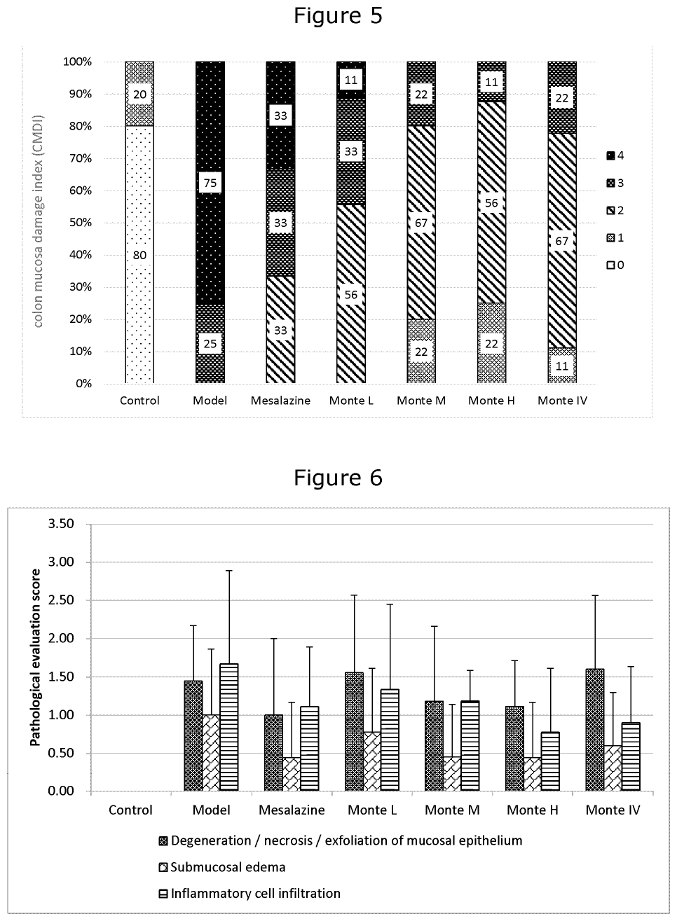

The invention is illustrated by the following examples, in which FIGS. 1 to 3 show the effect of montelukast on immune cell count in peripheral blood over time (FIG. 1), histopathological results (FIG. 2), and IL-1B concentrations in rectal tissues (FIG. 3) in irradiated rats; and FIGS. 4 to 7 show the effect of different drugs on colon-rectum function (FIG. 4), gross morphological evaluation of the colon mucosa (FIG. 5) and histopathological results (FIG. 6) in rats, in which radiation proctitis has been induced.

### EXAMPLE 1

### Radiation Proctitis in Rats

40 Sprague-Dawley rats were divided into 4 groups, each containing 10 rats.

Three groups (30 in total) of the rats were irradiated using a medical linear accelerator, with the 10 rats in a ‘normal control’ group were left untreated. The distance between the radiation source and the skin was 100 cm. The radiation field area was 2 cm×5 cm from the anus of rat and the radiation dose was 17.5 Gy. After irradiation, rats were put back to the cages.

A low dose (0.33 mg/g) montelukast gel was prepared by dissolving 0.033 g of montelukast sodium (Tianyu Pharmaceutical Co. Zhejiang, China) to 42.167 g of distilled water. 20.0 g of hydroxypropyl-beta-cyclodextrin (HP-β-CD, Shandong Binzhou Zhiyuan Biotechnology Co., Ltd.) was added slowly to the resultant solution with constant stirring until it dissolved completely. Then 24 g of hydroxypropyl methylcellulose (HPMC, Rohm Haas Electronic Materials (Shanghai) Co., Ltd.; 5% aqueous solution) was added and mixed well. 0.01 g of sodium hydroxide (China Pharmaceutical Group Chemical Reagents Co., Ltd) and 0.1 g EDTA-2Na (China Pharmaceutical Group Chemical Reagents Co., Ltd) were separately dissolved in 16.69 g distilled water to make the solution with pH of 7.2-7.5. The two solutions were mixed together with constant stirring and put aside until all of the bubbles disappeared.

Essentially the same procedure was employed to make a high dose (1 mg/g) montelukast gel by adding 0.1 g of montelukast sodium to approximately the same amount of distilled water and then adding the above-mentioned excipients in the same order.

The low dose and high dose montelukast gels were administered into rectums of each of 10 rats in two separate groups (‘low dose’ and ‘high dose’ groups, respectively) after irradiation. A blank gel base (the same gel as above but without montelukast) was given to the rats in the normal control group and the 10 remaining irradiated rats (‘model’ group).

Blood samples from the normal control, model and high dose groups were taken every other day. The total white blood cell, lymph cell and neutrophil counts were determined.

The results are shown in FIG. 1. Compared to the normal control group, the total cell counts were decreased significantly after irradiation. However, the cell count in high dose motelukast group started to increase after 5 days, which was not seen in the model group.

Rats were scarified after 7 days. 5 cm of rectal tissues were harvested and cut into two pieces, one part was sent for histopathological analysis (FIG. 2) and the other part was homogenized for cytokine detection (IL-1B) by ELISA kit (FIG. 3).

The results presented in FIG. 2 show that montelukast reduced the degree of damage (FIG. 2(a)) and promoted epithelial regeneration (FIG. 2(b)) in the rectum. The results presented in FIG. 3 show that the IL-1B concentrations in the model, low dose and high dose groups were decreased compared to the control group. However, the IL-1B concentrations in the montelukast treated groups were higher than that in the model group, in a dose dependent manner.

Thus, in a model that shows that, at levels of radiation that suppress the immune response of rats, montelukast can help to restore it, at the same time as promoting wound healing and having antiinflammatory effects.

### EXAMPLE 2

### Treating Radiation Proctitis by Transrectal and Intravenous Administration of Montelukast Gel

70 male Wistar rats weighing 180-220 g were obtained from Zhejiang Vital River Laboratory Animal Technology Co., Ltd (Zhejiang, China). All animals were maintained on standard rodent chow and tap water in standard cages with alternating 12-hour periods of light and darkness.

The rats were anesthetized with an intraperitoneal injection of 10% chloral hydrate (3.3 mL/kg). The rats were restrained and taped by the tail and four limbs on a cardboard in supine position. Irradiation was delivered using an Elekta Synergy medical linear accelerator (Elekta limited, UK). All animals except the sham operation group (‘Sham’) received a single continuous pelvic dose of irradiation. The distance from animal to source was 100 cm. The radiation area was 2 cm×5 cm, 5 cm upward from the anal orifice. The radiation dose was 17.5 Gy at a dose rate of 600 cGy/min.

After irradiation, the animals were put back into the cages for natural recovery. The animals in the Sham group were anesthetized in abdominal cavity without irradiation. Daily feed intake of rats and body weight were measured, and general observation were performed every day.

Day 1 (D1) was defined as the first day of drug administration, which was 24 hours after irradiation. Rats were given different drugs according to Table 1 below. Those in the Sham group and the model group (‘Model’) were given a blank gel (i.e. gel base prepared as described below, but without montelukast).

Sterile montelukast gels were prepared by mixing hydroxypropyl methylcellulose (24 mg), hydroxypropyl-beta-cyclodextrin (400 mg) and disodium edetate (2 mg) and sterilizing by steam at 121° C. for 30 minutes. 1 mg (Monte L), 3 mg (Monte M) or 10 mg (Monte H) of montelukast sodium was dissolved in water (1,564 mL) and was filtered through a 0.2 μm filter to sterilize it. The gels were then formed by mixing the two parts together.

A montelukast solution for intravenous (i.v.) injection (Monte IV) was made by dissolving 450 mg of montelukast sodium in 300 ml of water, which was then filtered through a 0.2 μm filter to obtain a sterilized 1.5 mg/mL.

Mesalazine suppositories (Dr Falk Pharma GmbH, Germany) were used as a positive control (Mesalazine). Suppositories were melted in 40° C. water bath, opened, and 0.2 g of mesalazine injected into the rectum of the rats.

Rats were treated once a day, and for 21 days continuously (D1 to D21).

TABLE 1

| Group | Treatment | Dose | | Volume | | Concentration | | Route |
|---|---|---|---|---|---|---|---|---|
| Control | Gel base | — | | 0.3 | g/rat | 0 | mg/g | rectal |
| Model | Gel base | — | | 0.3 | g/rat | 0 | mg/g | rectal |
| Mesalazine | from mesalazine | 45 | mg/rat | 0.2 | g/rat | 0.5 | g/piece | rectal |
| | suppositories | | | | | | | |
| Monte L | montelukast gel | 0.1 | mg/rat | 0.3 | g/rat | 0.33 | mg/g | rectal |
| Monte M | montelukast gel | 0.3 | mg/rat | 0.3 | g/rat | 1 | mg/g | rectal |
| Monte H | montelukast gel | 0.9 | mg/rat | 0.3 | g/rat | 3 | mg/g | rectal |
| Monte IV | montelukast | 3 | mg/kg | 2 | mL/kg | 1.5 | mg/ml | injection |
| | solution | | | | | | | |

To reduce bowel movement, and to extend the duration of the gel in the rectum, all animals were given a 6 mL/kg intraperitoneal injection of 5% chloral hydrate every day before administration. Formulations were introduced at about 3 cm inside the rectum by an intragastric needle, with the administration volume being 0.3 mL for each rat.

The overall state and stool characteristics were observed and recorded every day. A disease activity index (DAI) was evaluated according to the criteria described in Table 2 below. At day 22 (D22), all animals were sacrificed and rectum were harvested for evaluations. The rats were fasted for at least 12 hours in advance of administration.

TABLE 2

| Overall observation | | | | | |
|---|---|---|---|---|---|
| Abdominal | Stool | | | | Symptom |
| Distention (AD) | character | Death | Score | Grading | classification |
| AD | Normal | | 0 | <0.1 | Asymptomatic |
| AD | Very soft but | | 1 | <0.7 | Level 1 |
| | shaped | | | | |
| AD+ | Mucinous | | 2 | <1.3 | Level 2 |
| AD++ | Loose | | 3 | >2.0 | Level 3 |
| AD+++ | Loose (+) | | 4 | | |
| | Loose (++) | | 5 | | |
| | Loose (+++) | | 6 | | |
| | and/or blood | | | | |
| | | Death | 7 | | |

The rats were scarified by posterior carotid bloodletting after being anesthetized by an intraperitoneal injection of chloral hydrate

About 7 cm of the colorectal tract was separated at about 0.3 cm from the edge of perianal fur. The specimen was trimmed, and 1 cm of proximal and distal colorectal samples were cut off, respectively, by the same person. Then, the intestinal tube was dissected longitudinally, photographed and weighed.

Scores of the colon mucosa damage index (CMDI) were evaluated by gross observation according to the criteria in Table 3 below.

TABLE 3

| CMDI criteria | Score |
|---|---|
| Normal, no damage | 0 |
| Mild hyperemia, edema, smooth surface, no erosion | 1 |
| Moderate hyperemia, edema, rough granular mucosa with erosion or | 2 |
| intestinal adhesion. | |
| Highly hyperemia and edema with necrosis and ulcer on the mucosal | 3 |
| surface, the maximum longitudinal diameter of the ulcer is less than | |
| 1 cm, intestinal wall thickening or necrosis and inflammation on the | |
| surface. | |
| Based on 3 scores, the maximum longitudinal diameter of ulcer is | 4 |
| more than 1 cm or the whole intestinal wall is necrotic. | |

Specimens were fixed in 10% formaldehyde solution for 48 hours and stained with HE before examination with light microscopy by a pathologist (who was blinded to the study). Degeneration/necrosis/exfoliation of mucosal epithelium, submucosal edema and inflammatory cell infiltration were graded as follows:
0=normal or minor alterations which cannot be ascribed (with certainty) to radiation;
1=slight radiation damage (mild inflammation and/or slight crypt change);
2=mild damage (more significant inflammation, and/or crypt damage);
3=moderate damage (must have prominent loss of epithelium, degree of inflammation variable); and
4=severe damage (ulcers, necrosis).

The colon rectum function was evaluated by DAI scores, which are an indicator of colon-rectum function. The results are shown in Table 4 and in FIG. 4, in which the number of rats in different DAI levels in each group is shown.

TABLE 4

| Group | n | DAI score | Symptom grade |
|---|---|---|---|
| Control | 10 | 0.0 ± 0.03 | Asymptomatic |
| Model | 10 | 1.3 ± 1.01 | Level 2 |
| Mesalazine Suppositories | 10 | 1.0 ± 0.75 | Level 2 |
| Monte L | 10 | 0.9 ± 1.01 | Level 2 |
| Monte M | 10 | 0.6 ± 0.21 | Level 1 |
| Monte H | 10 | 0.5 ± 0.26 | Level 1 |
| Monte IV | 10 | 0.6 ± 0.74 | Level 1 |

Compared with the control groups, each of which showed various degree of sickness, such as diarrhea, soft, loose and/or mucinous stools, and even death, both intrarectal and intravenous administration of montelukast was found to reduce the severity of disease in a dose-dependent manner. Intravenous administration was slightly better than the Monte M intrarectal administration dose, but was less effective than Monte H dose.

The gross morphological evaluation of colon mucosa was evaluated by CMDI scores, in which higher scores represent a higher level of lesion. The results are shown Table 5 below and in FIG. 5, in which the percentage of CMDI score distribution in each group is shown.

TABLE 5

| Group | N | Mean | SD |
|---|---|---|---|
| Control | 10 | 0.2 | 0.42 |
| Model | 10 | 3.8 | 0.46 |
| Mesalazine | 10 | 3.0 | 0.87 |
| Monte L | 10 | 2.6 | 0.74 |
| Monte M | 10 | 2.0 | 0.67 |
| Monte H | 10 | 1.9 | 0.64 |
| Monte IV | 10 | 2.1 | 0.60 |

The results show that levels of lesions were reduced with increasing montelukast dose. Again, intravenous administration was slightly better than the Monte M dose, but was less effective than the Monte H dose, intrarectally administered.

The histopathological evaluation results are shown in FIG. 6, and show that montelukast gel reduced the lesions caused by radiation, with epithelial damage, submucosal edema and inflammatory cell infiltration being alleviated in a dose-related manner. Intrarectal administration showed better efficacy in repairing the epithelium than did intravenous administration.

1. The use of montelukast or a pharmaceutically acceptable salt thereof for the manufacture of a medicament for the treatment of an immunodeficiency disorder.

2. The use as claimed in claim 1, wherein the immunodeficiency disorder is a primary immunodeficiency disorder, selected from the group: a humoral immunodeficiency disorder, a cellular immunodeficiency disorder, a combined humoral and cellular immunodeficiency disorder, a phagocytic immunodeficiency and/or a complement deficiency.

3. The use as claimed in claim 1, wherein the immunodeficiency disorder is a secondary immunodeficiency disorder.

4. The use as claimed in claim 3, wherein the disorder is caused by one or more of the group: old age, malnutrition, a chronic disorder, one or more drugs and/or radiation.

5. The use as claimed in claim 4, wherein the chronic disorder causing immunosuppression is selected from the group: a cancer and/or a disorder of the blood selected from the group: aplastic anemia, a leukemia, multiple myeloma and sickle cell disease; Down's syndrome; a viral infection; a bacterial infection; diabetes mellitus; chronic kidney disease; nephrotic syndrome; chronic hepatitis; liver failure; systemic lupus erythematosus; alcoholism; a chronic burn and/or an operation.

6. The use as claimed in claim 4, wherein the one or more drugs is selected from the group: an antiseizure drug, an immunosuppressant a biologic, a chemotherapy drug and/or a corticosteroid.

7. The use as claimed in claim 4, wherein the radiation is radiation therapy administered during the treatment of a disorder.

8. The use of montelukast or a pharmaceutically acceptable salt thereof for the manufacture of a medicament for the treatment of a condition characterized by inflammation and/or wounding in a patient that has, or is vulnerable to, a suppressed immune system.

9. The use as claimed in claim 8 wherein the montelukast or pharmaceutically acceptable salt thereof provides an immunorestorative effect at the same time as promoting recovery and/or healing of wounds that are associated with said condition.

10. The use as claimed in claim 8 or claim 9, wherein the montelukast or pharmaceutically acceptable salt thereof provides an antiinflammatory effect without compromising the patient's immune system.

11. The use as claimed in any one of claims 8 to 10, wherein the immunosuppression is brought on by radiation therapy administered during the treatment of a disorder.

12. The use as claimed in claim 7 or claim 11, wherein the disorder being treated by the radiation therapy is cancer.

13. The use as claimed in any one of claim 7, 11 or 12, wherein the radiation therapy is targeted to the lower abdomen.

14. The use as claimed in any one of claims 8 to 13, wherein the condition characterized by inflammation is selected from the group: radiation enteritis, radiation colitis, radiation hepatitis, radiation myelitis, radiation vaginitis and radiation proctitis.

15. The use as claimed in claim 14, wherein the disorder is radiation proctitis and/or comprises one or more of the group: acute radiation proctitis, radiation colitis, radiation associated vascular ectasias and/or chronic radiation proctopathy.

16. The use as claimed in any one of the preceding claims wherein the montelukast or pharmaceutically acceptable salt thereof is applied locally and topically.

17. The use as claimed in claim 16 wherein the montelukast is applied in the form of a gel.

18. The use as claimed in claim 16 or claim 17, wherein the local, topical application is anorectal.

19. Montelukast or a pharmaceutically acceptable salt thereof for use in a method of treatment of a disorder as defined in any one of claims 1 to 15.

20. A method of treatment of a disorder as defined in any one of claims 1 to 15, which method comprises the administration of montelukast or a pharmaceutically acceptable salt thereof to a patient in need of said treatment.

21. A method of treatment of a condition characterized by immunosuppression in a patient, which method comprises the administration of montelukast or a pharmaceutically acceptable salt thereof to a patient in need of said treatment.

22. A method as claimed in claim 21, wherein the condition is a disorder as defined in any one of claims 1 to 15.

23. A method of treatment of a patient with a compromised immune system, which method comprises the administration of montelukast or a pharmaceutically acceptable salt thereof to such a patient.

24. A method for restoring a normal function of the immune system of a patient, which method comprises the administration of montelukast or a pharmaceutically acceptable salt thereof to such a patient.

25. A method of treatment of a radiation-induced condition that is characterized by immunosuppression, as well as inflammation and/or wounding, in a patient, which method comprises the administration of montelukast or a pharmaceutically acceptable salt thereof to a patient in need of said treatment.

26. A method of treating inflammation and/or wounds that are associated with a radiation-induced immunodeficiency disorder whilst simultaneously restoring the normal function of the immune system of a patient, which method comprises the administration of montelukast or a pharmaceutically acceptable salt thereof to a patient in need of said treatment.

27. A compound for use as claimed in claim 19, or method as claimed in any one of claims 20 to 26, wherein the method comprises administering montelukast or pharmaceutically acceptable salt thereof in a manner as defined in any one of claims 16 to 18.

28. A use, compound for use, or a method, as claimed in any one of the preceding claims, wherein the pharmaceutically acceptable salt of montelukast is montelukast sodium.
